(19) United States
(12) Patent Application Publication
(10) Pub. No.: US 2010/0030005 A1
(43) Pub. Date: Feb. 4, 2010

(54) PROCESSES FOR PRODUCING HIGHER HYDROCARBONS FROM METHANE AND BROMINE

(76) Inventors: Joe D. Sauer, Baton Rouge, LA (US); George W. Cook, Baton Rouge, LA (US); Tyson J. Hall, Baton Rouge, LA (US); Bonnie Gary McKinnie, Magnolia, AR (US)

Correspondence Address: ALBEMARLE CORPORATION;PATENT DEPARTMENT, 451 FLORIDA STREET, BATON ROUGE, LA, 70801, US

(21) Appl. No.: 12/528,241

(22) PCT Filed: Feb. 13, 2008
(86) PCT No.: PCT/US08/53806

§ 371 Date: Aug. 21, 2009

Related U.S. Application Data
(60) Provisional application No. 60/905,007, filed on Feb. 28, 2007.

(30) Foreign Application Priority Data
Dec. 19, 2007 (US) 61014865

(51) Int. Cl. C07C 2/00

(52) U.S. Cl. 585/700

(57) ABSTRACT

Processes for producing C2+ hydrocarbons are provided. Such processes use Br2, HBr, and/or heat that are produced by such processes, thus providing commercially efficient processes. The process can comprise (a) producing HBr and methyl bromide using a bromine source and a gas stream comprising methane; (b) heating the methyl bromide in the presence of a catalyst to produce additional HBr and C2+ hydrocarbons; (c) combining at least some of the HBr and an oxygen source in the presence of a cerium-containing compound at least about 315° C. to produce Br2; and (d) using at least some of the produced Br2 from (c) as at least a portion of the bromine source in (a). Additionally, the additional HBr from (b) can be used in (c) and/or heat can be recovered from (c) and used to provide at least some of the heating in (a), (b), or both.

### BACKGROUND

Methane is a major constituent of natural gas and also of biogas. World reserves of natural gas are constantly being increased, e.g., due to new discoveries, etc. However, a significant portion of the world reserves of natural gas is in remote and offshore locations where gas pipelines cannot be economically justified or reinjection of the gas is not feasible. Thus, much of the natural gas produced along with oil at remote locations is flared. The same is true of methane produced in petroleum refining and petrochemical processes. Since flaring of methane produces CO₂, future flaring of natural gas and methane may be prohibited or restricted. Thus, significant amounts of natural gas and methane are available to be utilized.

Different technologies have been described for utilizing these sources of natural gas, including methane. For example, technologies are available for converting natural gas to higher hydrocarbons, i.e., liquids fuels, which are more easily transported than natural gas. Alternatively, methane can be sweetened, dried, and transported to market; however the sweetened and dried methane product is typically sold at ½ to ⅓ the price of liquid fuels on a BTU basis.

In regard to converting natural has to liquid fuels, the Fischer Tropsch (FT) reaction involves the synthesis of liquid hydrocarbons or their oxygenated derivatives from the mixture of carbon monoxide and hydrogen, which can be obtained, e.g., by the partial combustion of methane or by the gasification of coal. This synthesis is carried out with metallic catalysts such as iron, cobalt, or nickel at high temperature and pressure. The overall efficiency of the FT reaction and subsequent water gas shift chemistry is estimated at about 15% to 30%, when allowing for the energy required to make the conversion. While FT does provide a route for the liquefication of coal stocks, it is not adequate in its present level of understanding and production for commercial conversion of methane-rich stocks to liquid fuels. FT requires a heavily discounted natural gas source to be economical. Additionally, a FT plant is expensive and bulky, and therefore not suitable for use in many remote locations, such as on an offshore oil rig where natural gas comprising methane is routinely flared.

Osterwalder and Stark (“O/S”) have proposed a method for the direct coupling of bromine-mediated methane activation and carbon-deposit gasification in the conversion of methyl bromide into light hydrocarbons (e.g., Chem Phys Chem 2007, 8(2), 297-303). Referring to FIG. 1 (PRIOR ART), which is a process diagram provided by O/S, a method for the direct coupling of bromine-mediated methane activation and carbon-deposit gasification is proposed. It can be seen that, in the proposed process, methane streams 10a and 10b and bromine stream 20 are combined for an alkane bromination step 30, which produces a methyl bromide stream 40 and a hydrogen bromide stream 50. As shown, the methyl bromide stream 40 and a methyl bromide recycling stream 100 are combined in the presence of aluminum bromide for alkyl bromide conversion 70 to produce a an output stream 72. The output stream 72 is separated in separator 75 into higher hydrocarbon (C₂-C₅) product stream 80, the methyl bromide recycling stream 100, an HBr stream 90, and the methane stream 10b.

While O/S shows the HBr streams 50 and 90 going to a bromine recycling step 60 to produce bromine stream 20, the mechanics and other details of the bromine recycling step are not taught or suggested. At least about ten pounds of bromine are required for every pound of higher hydrocarbon product. Given the substantial amounts of natural gas and methane available for conversion to higher hydrocarbons, and the diversity, and often remoteness, of locations at which such natural gas/methane is available, in order for the O/S method, or any similar method of converting methane to higher hydrocarbons, to be commercially feasible, a means for recovering bromine from HBr for reuse in the conversion method must be provided. An efficient bromine recycle procedure could make large-scale methane to liquid processing plants economically feasible.

Processes for production of bromine from bromide-containing solutions such as brines are know. For example, bromine can be produced by a bromine steaming out process, such as Kubierschky's distillation method; see, e.g., Kirk-Othmer, Encyclopedia of Chemical Technology, Fourth Edition, volume 4, pages 548 through 553. Other methods for recovering bromine from bromide-containing solutions are described, e.g., in U.S. Pat. No. 3,181,934, U.S. Pat. No. 4,719,096, U.S. Pat. No. 4,978,518, U.S. Pat. No. 4,725,425, U.S. Pat. No. 5,158,683, and U.S. Pat. No. 5,458,781. Otherwise, bromine can be recovered from brines by treatment with chlorine to oxidize the bromide to bromine; and processes for electrolytic conversion of bromide to bromine are known. Additionally, catalytic oxidation of bromide to bromine by use of oxygen or air mixtures has been reported (see, e.g., U.S. Pat. No. 5,366,949); however, to our knowledge no successful, economic, commercial operation is in place today.

In spite of technologies that are currently described and available, to our knowledge there are no commercial or reported processes for conversion of alkanes to useful hydrocarbons that include a suitable process for converting bromide to bromine for use in the conversion process. It would be commercially beneficial if such processes were available.

### THE INVENTION

This invention meets the above-described needs by providing processes for producing C2+ hydrocarbons, such process comprising: (a) producing HBr and methyl bromide using a bromine source and a gas stream comprising methane; (b) heating at least some of the methyl bromide in the presence of a catalyst to produce additional HBr and C2+ hydrocarbons; (c) processing at least some of the HBr to produce Br₂; (d) using at least some of the produced Br₂ from (c) as at least a portion of the bromine source in (a). This invention provides such processes wherein: the processing of (c) comprises combining at least some of the HBr and an oxygen source in the presence of a cerium-containing compound at at least about 315° C. to produce Br₂; the processing of (c) comprises combining at least some of the HBr, at least some of the additional HBr, and an oxygen source in the presence of a cerium-containing compound at at least about 315° C. to produce Br₂; and/or wherein such processes comprise recovering heat from (c) and using the recovered heat to provide at least some of the heating in (a), (b), or both. As used herein the term C2+ hydrocarbons includes all hydrocarbons having two or more carbon atoms, including without limitation ethane, propane, butane, ethylene, propene, heptane, isooctane, cyclopentane, ethyl benzene, and the like.

When processing at least some of the HBr to produce Br₂ comprises combining at least some of the HBr and an oxygen source in the presence of a cerium-containing compound at at least about 315° C. to produce Br₂; the processing can be conducted, e.g., at at least about 315° C. (600° F.) to about 1000° C. (1832° F.), or at at least about 315° C. (600° F.) to about 538° C. (1000° F.). As will be familiar to those skilled in the art, the upper temperature can be limited by the ability of the cerium-containing compound, or other catalyst, and/or of the processing equipment to withstand the temperature of operation.

While the description provided herein focuses on HBr oxidation in the presence of a cerium-containing compound, e.g., a cerium-based catalyst, other processes for obtaining Br₂ from HBr are suitable for use in processes of this invention. For example, processes whereby HBr is treated electrolytically to generate hydrogen and bromine (Br₂) may be used.

Other processes useful in processes of this invention include, for example, reacting HBr and methane at elevated temperatures in an oxygen atmosphere in the presence of a lanthanum catalyst to generate methyl bromide and water. The methyl bromide can be converted to C2+ hydrocarbons and/or other organic products, generating HBr as a co-product. In applications where methanol is available, methanol and HBr can be used to generate methyl bromide and water. The methyl bromide can be converted to C2+ hydrocarbons and/or other organic products, generating HBr as a co-product. The co-product HBr can be recycled for use in processes of this invention.

### FIGURES

The invention will be better understood by reference to the Figures wherein:

FIG. 1 (PRIOR ART) illustrates a method for the direct coupling of bromine-mediated methane activation and carbon-deposit gasification; and

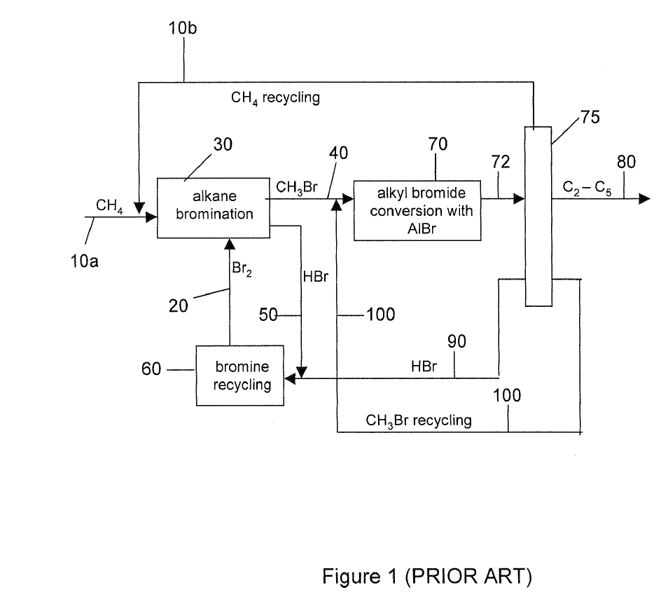

FIG. 2 is a flow diagram representative of an exemplary process according to this invention.

The descriptions in this specification are illustrative of the principles of this invention. This invention is not limited to any one specific embodiment exemplified herein, whether in the Figures, the examples or the remainder of this patent application.

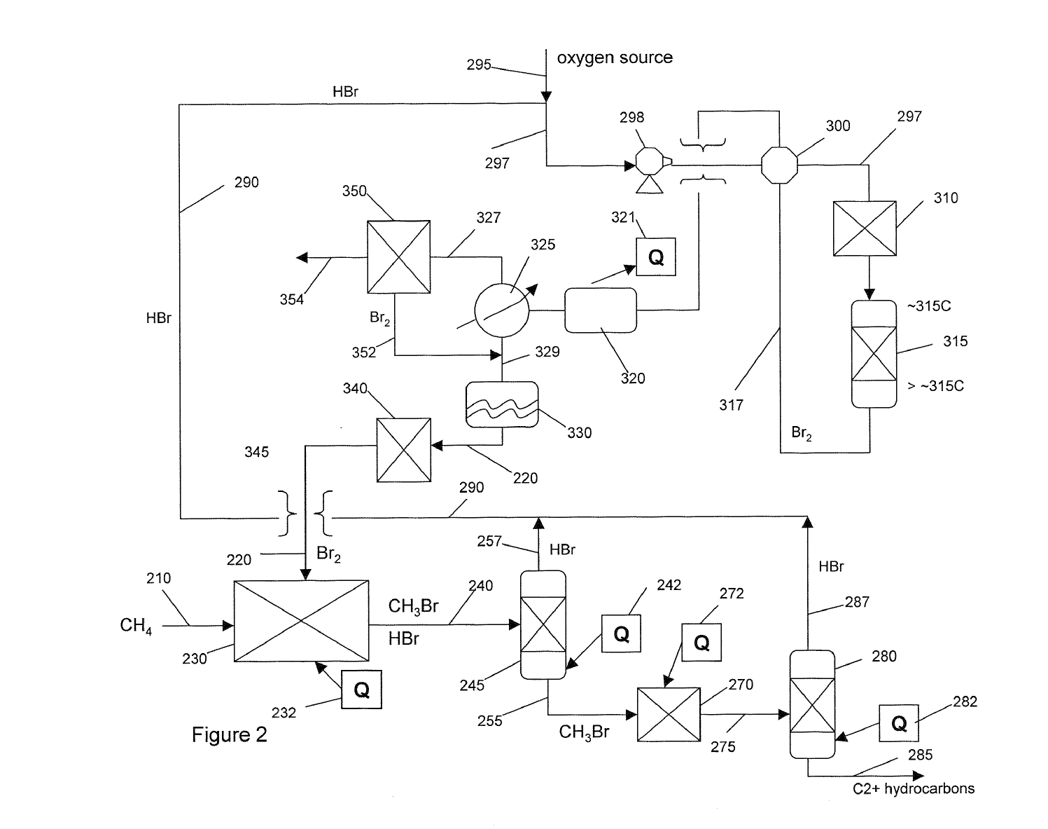

Referring to FIG. 2, in an example process according to this invention, methane stream 210 and bromine stream 220 can be combined for alkane bromination 230, to produce a stream 240 comprising methyl bromide and hydrogen bromide. Alkane bromination 230 is endothermic and requires heat that can be supplied by heat source 232. In separation 245, stream 240 can be separated into HBr stream 257 and methyl bromide stream 255. Heat source 242 can be used for heating in separation 245. Methyl bromide stream 255 can be heated in the presence of an aluminum halide, or other suitable catalyst, for alkyl bromide conversion 270 to produce a product stream 275. Heat source 272 is used for heating in alkyl bromide conversion 270. Product stream 275 can be separated in separation 280 into product stream 285 comprising C2+ hydrocarbons and HBr stream 287. Heat source 282 can be used for heating in separation 280.

In this process, HBr streams 257 and 287 can be combined into HBr stream 290, which can be combined with oxygen source stream 295 into stream 297 which can be blown via blowing device 298 through heat interexchanger 300. Oxygen source stream 295, and thus stream 297, comprises oxygen and can comprise many inerts including nitrogen, argon, carbon dioxide, neon, etc. A start-up furnace 310 can provide initial heating, and supplemental heating as needed, to heat stream 297 to at least about 315° C. (600° F.). Heated stream 297 can be input to reactor 315 containing a cerium-containing compound. HBr in stream 297 can be oxidized in an exothermic reaction in reactor 315. Stream 317 exiting reactor 315 at a temperature higher than about 315° C. (600° F.) to at least about 427° C. (800° F.), can comprise Br₂, H₂O, and inerts, and can be passed through heat interexchanger 300 for providing heating and through waste heat boiler 320 for recovery of recovered heat 321. Stream 317 can then be input to condenser 325 for separation into (i) stream 327 that comprises Br₂ and can comprise inerts and (ii) stream 329 comprising Br₂ and H₂O. Stream 327 can be input to bromine scrubber 350 for separation into stream 352 comprising Br₂ and stream 354 that can comprise inerts. Stream 352 can be combined with stream 329 either before (as shown) or after entry of stream 329 into separator 330. Br₂ recovered from separator 330 in stream 220 can be dried in dryer 340 and combined with stream 210 for alkane bromination 230. Recovery of water from separator 330 is not shown in the Figure.

Recovered heat 321 can be used to provide heat as needed in processes of this invention, e.g., can be used to provide and/or supplement the heat in heat source 232, heat source 242, heat source 272, and/or heat source 282. Start-up furnace 310, or any other suitable heat source, e.g., steam, can provide start-up and/or supplemental heat.

The oxygen source in processes of this invention can comprise oxygen and other components, including without limitation, nitrogen, argon, and carbon dioxide, and can comprise air. Excess air can be used.

Heat generated and used herein can come from any suitable source, as will be familiar to those skilled in the art. For example, geothermal steam can be used. Also, water can be heated to form steam by any suitable heating means, as will be familiar to those skilled in the art. As used herein, steam comprises H₂O and can comprise other components. Both direct and indirect heating can be used in processes of this invention.

Cerium-containing compounds useful in alkyl bromide conversion in processes of this invention can be any suitable cerium-containing compound. Such cerium-containing compounds are used as catalysts. Suitable catalysts are described, e.g., in U.S. Pat. No. 5,366,949 (Schubert), and include cerium bromide, cerium oxide, and the like. A suitable catalyst composition can comprise cerium bromide on zirconia containing supports.

Residence time of heated HBr and oxygen inside of a reactor can vary depending on factors such as the size of the reactor, whether the contents of the reactor are under pressure, etc., as will be familiar to those skilled in the art.

Unless otherwise specified herein, streams described as comprising specified components may also comprise additional components including without limitation HCl, Cl₂, CO₂, and unreacted HBr.

Those skilled in the art will appreciate that, in particular in heat exchange equipment used in processes of this invention, in order for such processes to b commercially applicable, materials of construction should be suitable for holding up under the pressures, temperatures, and other conditions to which the equipment will be subjected. Some suitable materials where the temperature is less than about 204° C. (400° F.) include Ta and Zr, and Ti when water is present. Some equipment, e.g., reactors, may be constructed from corrosion resistant materials, or may have a corrosion resistant lining. For example, a reactor can be constructed from quartz or acid brick, or can be constructed to have a refractory or zirconia lining. Care should be taken when heating and cooling equipment not to shock the equipment such that cracks are started.

Processes of this invention are particularly well suited for improving commercial/economic feasibility of large-scale natural gas/methane to liquid processing plants.

It is to be understood that the reactants and components referred to by chemical name or formula anywhere in the specification or claims hereof, whether referred to in the singular or plural, are identified as they exist prior to being combined with or coming into contact with another substance referred to by chemical name or chemical type (e.g., another reactant, a solvent, or etc.). It matters not what chemical changes, transformations and/or reactions, if any, take place in the resulting combination or solution or reaction medium as such changes, transformations and/or reactions are the natural result of bringing the specified reactants and/or components together under the conditions called for pursuant to this disclosure. Thus the reactants and components are identified as ingredients to be brought together in connection with performing a desired chemical reaction or in forming a combination to be used in conducting a desired reaction. Accordingly, even though the claims hereinafter may refer to substances, components and/or ingredients in the present tense (“comprises”, “is”, etc.), the reference is to the substance, component or ingredient as it existed at the time just before it was first contacted, combined, blended or mixed with one or more other substances, components and/or ingredients in accordance with the present disclosure. Whatever transformations, if any, which occur in situ as a reaction is conducted is what the claim is intended to cover. Thus the fact that a substance, component or ingredient may have lost its original identity through a chemical reaction or transformation during the course of contacting, combining, blending or mixing operations, if conducted in accordance with this disclosure and with the application of common sense and the ordinary skill of a chemist, is thus wholly immaterial for an accurate understanding and appreciation of the true meaning and substance of this disclosure and the claims thereof. As will be familiar to those skilled in the art, the terms “combined”, “combining”, and the like as used herein mean that the components that are “combined” or that one is “combining” are put into a container with each other. Likewise a “combination” of components means the components having been put together in a container.

While the present invention has been described in terms of one or more preferred embodiments, it is to be understood that other modifications may be made without departing from the scope of the invention, which is set forth in the claims below. Particularly, this invention is suitable for providing bromine recycle capabilities to all types of processes for converting natural gas/methane to C2+ hydrocarbons.

What is claimed is:

1. A process for producing C2+ hydrocarbons, the process comprising:
(a) producing HBr and methyl bromide using a bromine source and a gas stream comprising methane;
(b) heating at least some of the methyl bromide in the presence of a catalyst to produce additional HBr and C2+ hydrocarbons;
(c) combining at least some of the HBr and an oxygen source in the presence of a cerium-containing compound at at least about 315° C. to produce Br₂;
(d) using at least some of the produced Br₂ from (c) as at least a portion of the bromine source in (a).

2. The process of claim 1 wherein (c) is replaced with:
(c) combining at least some of the HBr, at least some of the additional HBr, and an oxygen source in the presence of a cerium-containing compound at at least about 315° C. to produce Br₂.

3. The process of claim 1 further comprising recovering heat from (c) and using the recovered heat to provide at least some of the heating in (a), (b), or both.
